(19) United States
(12) Patent Application Publication
(10) Pub. No.: US 2006/0089683 A1
(43) Pub. Date: Apr. 27, 2006

(54) Transcutaneous nerve and muscle stimulator and method of using the same

(76) Inventors: James T. Hagglof, Monument, CO (US); Chad E. Abercrombie, Monument, CO (US); Scott D. Rosenquist, Colorado Springs, CO (US)

Correspondence Address: PROCOPIO, CORY, HARGREAVES & SAVITCH LLP, 530 B STREET, SUITE 2100, SAN DIEGO, CA, 92101, US

(21) Appl. No.: 11/296,981

(22) Filed: Dec. 7, 2005

Related U.S. Application Data
(62) Division of application No. 10/337,572, filed on Jan. 7, 2003.
(60) Provisional application No. 60/427,866, filed on Nov. 19, 2002.
(60) Provisional application No. 60/432,180, filed on Dec. 10, 2002.

(51) Int. Cl. A61N 1/08

(52) U.S. Cl. 607/48

(57) ABSTRACT

A transcutaneous electrical stimulator system includes a small, lightweight, portable, external, programmable transcutaneous electrical stimulator that provides multiple electrical stimulation therapies to a patient through the patient's skin. The stimulator includes persistent, modifiable memory programmable by a practitioner for prescribing multiple electrical stimulation therapies for the patient. A programming pod is configured to interface with the stimulator and a computer to program multiple stimulation therapies into the persistent, modifiable memory of the stimulator.

### CROSS-REFERENCE TO RELATED APPLICATION

This application is a divisional of U.S. patent application Ser. No. 10/337,572, filed Jan. 7, 2003, which claims priority of provisional application No. 60/427,866 filed Nov. 19, 2002 and provisional application No. 60/432,180 filed Dec. 10, 2002, all hereby incorporated in their entirety by reference.

### FIELD OF THE INVENTION

The present invention is, in general, in the field of medical transcutaneous stimulators that provide an electrical stimulation signal in the form of a continuous or interrupted train of pulses to a patient for nerve and muscle stimulation, and edema reduction.

### BACKGROUND OF THE INVENTION

Medical stimulators that provide electrical stimulation signals to a patient are used to provide short and long term pain relief through transcutaneous electrical nerve stimulation (“TENS”) and to stimulate and rehabilitate muscles through neuromuscular stimulation (“NMS” or “EMS”). As used herein, a “transcutaneous electrical stimulator” includes at least a TENS stimulator, a NMS or EMS stimulator, a stimulator used for edema reduction or any other medical stimulator used to transcutaneously deliver therapeutic electrical impulses. These types of medical stimulators typically include lead wires with distal electrodes that are attached to the patient's skin. The transcutaneous electrical stimulator sends electrical stimulation signals into the muscles and nerves though the attached electrodes. The electrical stimulation signals produced by the transcutaneous electrical stimulator are in the form of a train of electrical pulses which may be modulated in rate and/or intensity.

Transcutaneous electrical stimulators may use a periodic treatment mode and/or a continuous treatment mode. The periodic treatment mode includes an on time cycle and an off time cycle. During the on time cycle, a train of pulses forming the stimulation signal is delivered, and during the off time cycle, no pulses are delivered. The continuous treatment mode includes a continuous train of pulses provided as output.

A problem with conventional transcutaneous electrical stimulators is that they include controls on the stimulators for setting the proper and unique operation of the transcutaneous electrical stimulator. Some patients may try to alter the controls without understanding the effect of such alterations, causing ineffective or destructive electrical stimulation signals to be delivered.

Another problem with conventional transcutaneous electrical stimulators is that they deliver electrical stimulation signal patterns, treatment periods, and frequencies inappropriate for the complexity of the muscles.

A further problem is that transcutaneous electrical stimulators do not allow for fast, easy programming of appropriate electrical stimulation signal patterns, treatment periods, and frequencies in the transcutaneous electrical stimulator.

A still further problem with transcutaneous electrical stimulators is that they tend to be rather large and bulky, making them uncomfortable to wear. Transcutaneous electrical stimulators can also be very time intensive for patients if they need to visit a practitioner on a frequent basis for treatment using a transcutaneous electrical stimulator.

### SUMMARY OF THE INVENTION

An aspect of the invention involves a transcutaneous electrical stimulator system for use with a computer. The transcutaneous electrical stimulator system includes a small, lightweight, portable, external, programmable transcutaneous electrical stimulator that provides multiple electrical stimulation therapies to a patient through the patient's skin. The stimulator includes persistent, modifiable memory programmable by a practitioner for prescribing multiple electrical stimulation therapies for the patient. A programming pod is configured to interface with the stimulator and the computer to program multiple stimulation therapies into the persistent, modifiable memory of the stimulator. One of many unique firmware executables (standard, special, or custom) may also be programmed into memory of the stimulator. Each unique firmware executable may include its own protocol for sequence of therapies and/or intensities.

Another aspect of the invention involves a method of programming multiple electrical stimulation therapies into a transcutaneous electrical stimulator at a practitioner location. The method includes providing at the practitioner location a small, lightweight, portable, external, programmable transcutaneous electrical stimulator that provides multiple electrical stimulation therapies to a patient through the patient's skin, the stimulator including persistent, modifiable memory programmable by a practitioner for prescribing multiple electrical stimulation therapies for the patient; providing at the practitioner location a programming pod interfaced with the stimulator to program multiple stimulation therapies into the persistent, modifiable memory of the stimulator; providing at the practitioner location a computer interfaced with the programming pod to program multiple stimulation therapies into the persistent, modifiable memory of the stimulator via the programming pod; and using the computer at the practitioner location to program multiple therapies into the persistent, modifiable memory of the stimulator via the programming pod.

A further aspect of the invention involves a computer readable medium having stored thereon one or more sequences of instructions for causing one or more microprocessors to perform the steps for programming multiple electrical stimulation therapies into a transcutaneous electrical stimulator via a programming pod. The steps include selecting a specific therapy to be programmed out of multiple sequential electrical stimulation therapies; selecting one of multiple different types of predetermined therapies and a custom therapy; selecting a type of polarity for the stimulator; selecting a time duration of the therapy; selecting an idle time duration between therapies; selecting the next specific therapy to be programmed out of multiple sequential electrical stimulation therapies and repeating the above steps until all the therapies of the multiple sequential electrical stimulation therapies are input; selecting one of a repeat function to cause the stimulator to cycle through multiple therapies then repeat and a terminate function to cause the stimulator to cycle through multiple therapies then stop; and selecting a program function to cause the programming pod to program the multiple electrical stimulation therapies into the transcutaneous electrical stimulator.

### BRIEF DESCRIPTION OF THE DRAWINGS

FIG. 1 is a schematic diagram of an embodiment of a transcutaneous electrical stimulator system, a computer, and a printer.

FIG. 2A is a front perspective view of an embodiment of a small, portable, external, programmable transcutaneous electrical stimulator that may be used as part of the system illustrated in FIG. 1.

FIG. 2B is a rear perspective view of the stimulator of FIG. 2A.

FIG. 2C is a front perspective view of an embodiment of self-adhesive electrodes that may be used with the stimulator of FIG. 2A.

FIG. 2D is a front perspective view of an embodiment of a docking pod or cradle that may be used with the stimulator of FIG. 2A.

FIG. 2E is a block diagram of an embodiment of electronics that may be used in the stimulator of FIG. 2A.

FIG. 2F is a block diagram of an embodiment of electronics that may be used in the docking pod of FIG. 2D.

FIG. 3 illustrates waveforms of an example transcutaneous stimulation therapy and waveforms of a recharge interval during which the stimulator may be recharged.

FIG. 4 is a table illustrating an embodiment of therapy parameters that may be programmed into the stimulator.

FIG. 5 is a table illustrating exemplary locations in EEPROM memory where information on therapy parameters may reside.

FIGS. 6-8 illustrate an exemplary dialog box that may be used in the programming of the stimulator, and show fields for text entry or therapy parameter selection.

FIG. 9 is a block diagram illustrating an exemplary computer as may be used in connection with various embodiments described herein.

### DETAILED DESCRIPTION OF PREFERRED EMBODIMENTS

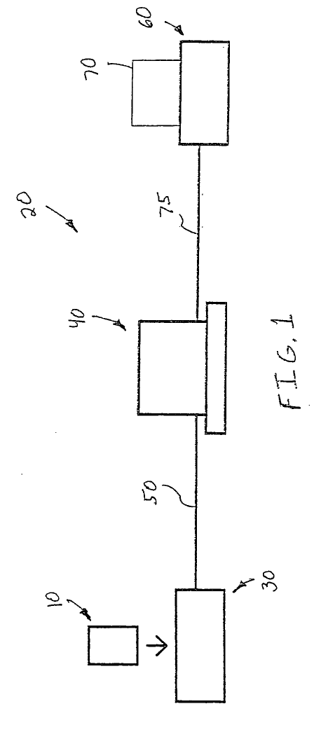

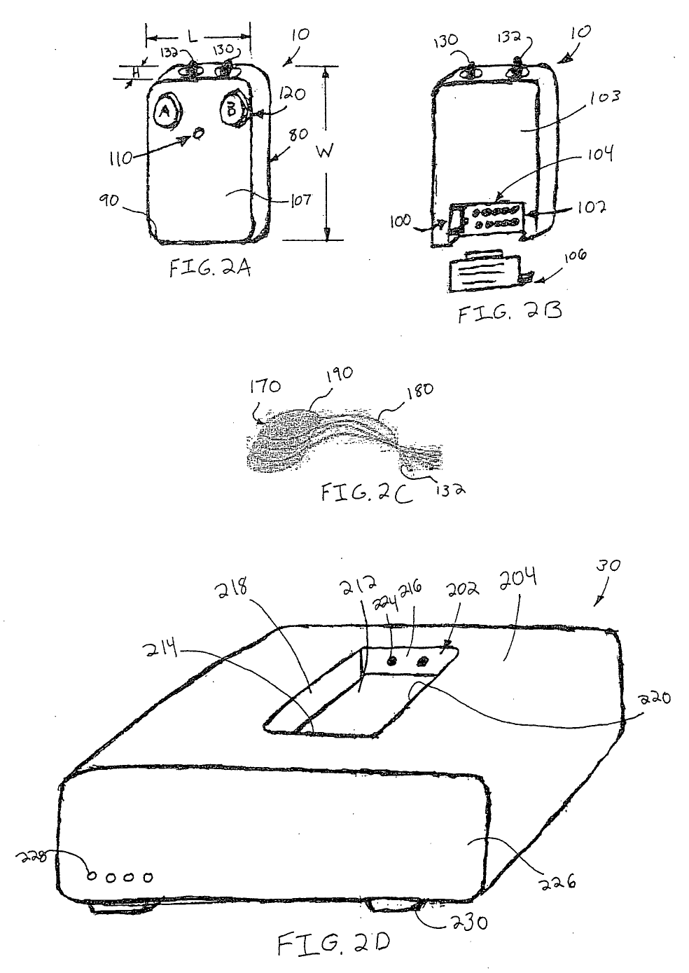

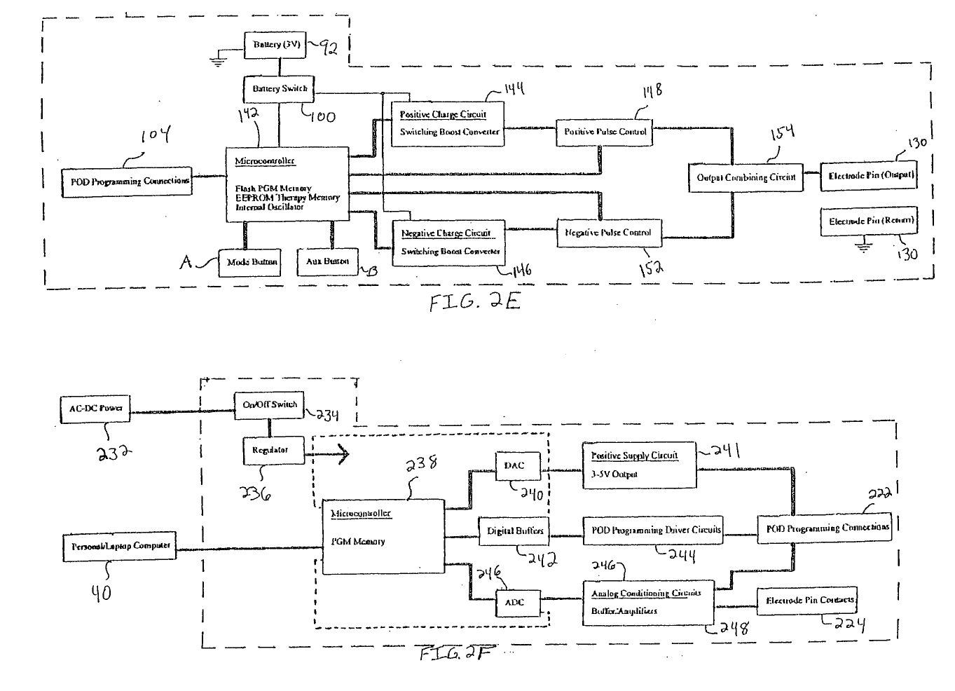

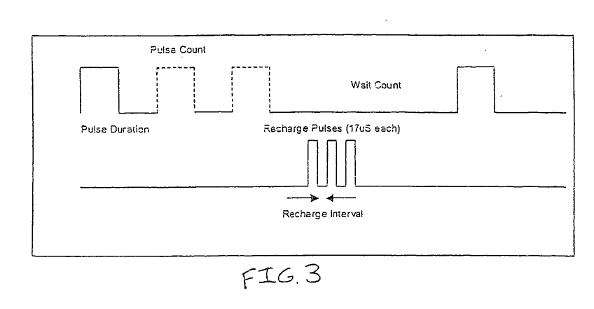

With reference initially to FIGS. 1 and 2, a small, lightweight, portable, external, programmable transcutaneous electrical stimulator (hereinafter “stimulator”) 10 constructed in accordance with an embodiment of the invention and which may be used as part of a transcutaneous electrical stimulator system 20 will be described. The stimulator 10 may be used externally to provide electrical stimulation signals to a patient. As used herein, “external” or “externally” means the stimulator 10 is non-implantable and the electrical stimulation signals are delivered from outside the patient's body (i.e. typically via one or more electrodes affixed to the patient's skin) transcutaneously, through the patient's skin. The electrical stimulation signals may be used to provide short and long term pain relief through transcutaneous electrical nerve stimulation (“TENS”), to stimulate and rehabilitate muscles through neuromuscular stimulation (“NMS”), and/or to provide pseudo-random bipolar low-intensity micro-current stimulation for edema reduction.

Before specifically describing the stimulator 10, the overall system 20 will be described generally. The system 20 includes the stimulator 10 and a docking station or pod 30 residing at a medical practitioner's office. The pod 30 may be communicatively coupled to a personal or laptop computer 40 (hereinafter “computer”) through one or more connections 50 such as, but not by way of limitation, a serial connection (e.g., RS232, USB), a parallel connection, and/or network. One or more output devices at the practitioner's office such as a printer 60 for printing reports 70 related to the stimulator 10 may be communicatively coupled to the computer 40 through one or more connections 75 such as, but not by way of limitation, a parallel connection and/or a network. The computer 40 may be used to program one or more electrical stimulation therapies and/or operating software (or operating software upgrades) into the stimulator 10 (via the pod 30). The computer 40 and the pod 40 may also be used to receive data from the stimulator 10 such as, but not limited to, a summary report of the performance history of the stimulator 10 since the last programming/docking, any error codes that may have occurred during performance of the stimulator 10, any checksum discrepancies that may have occurred during performance of the stimulator 10, battery power level in the stimulator 10, and stimulator diagnostic and verification information.

With reference to FIGS. 2-5, and initially to FIG. 2A, the stimulator 10 and pod 30 will be described in more detail. In the embodiment shown, the stimulator 10 has a generally rectangular block-shaped housing 80. Corners 90 of the housing 80 may be rounded to give the stimulator 10 a more rounded ergonomic look and feel. In a preferred embodiment, the stimulator 10 is much smaller and lighter than TENS devices used in past (TENS devices used in the past were generally about the size of a Sony® Walkman® cassette player; the batteries used in the prior art TENS devices were often larger than the stimulator 10 described herein) and includes a length L of 1.05 in., a width W of 1.65 in., and a height H of 0.30 in. The stimulator 10 is considerably smaller, lighter, less cumbersome, and less noticeable to the user or patient than stimulators used in the past, increasing the comfort of the patient and increasing the chances that the patient will use the stimulator 10. In alternative embodiments, the stimulator 10 may include one or more dimensions smaller than those specified above.

The stimulator 10 may operate on a single, disposable, very small 3V lithium coin cell battery 92 (FIG. 2E). Although a single coin cell 3V battery is described, other power sources, smaller power sources, or other numbers of power sources may be used. Because of the prescription nature of the programmable stimulator 10, the battery is preferably disposable and not replaceable by the patient.

In an embodiment of the invention, the stimulator 10 is returned to the practitioner upon a return visit or appointment with the practitioner. The stimulator 10 may then be sent by the practitioner to a third party, where the stimulator 10 is refurbished. Refurbishing of the stimulator 10 by the third party may include removal and responsible environmental disposal of the battery, replacement of the battery with a new battery, and stimulator integrity verification. The stimulator 10 may then be sold or shipped to another practitioner for prescription programming/reprogramming and use with another user or patient.

In another embodiment of the invention, the stimulator 10 is returned to the practitioner upon a return visit or appointment with the practitioner. The stimulator 10 may be refurbished by the practitioner by replacing the battery. The stimulator 10 may be reprogrammed and/or integrity verified, and then issued to the same or a different user or patient.

The stimulator 10 may provide unipolar (positive or negative) or bipolar (positive and negative) pulsed energy waveforms for localized pain control or muscle stimulation. In a preferred embodiment, the only patient-controllable input on the stimulator 10 may be an “A” button on a front 107 of the stimulator 10 that toggles the current programmed therapy on or off, and a “B” button that may be pressed while the stimulator 10 is in an active mode to display the programmed therapy number via a mode indicator 110 such as a green LED. Once the “B” button is pressed, the indicator 110 may flash a number of times associated with the programmed therapy number (e.g., when the stimulator is on therapy four, the indicator 110 may flash four times). The “A” button may also be used for controlling the mode of the stimulator 10 and the “B” button may be used for controlling auxiliary functions once the stimulator 10 is in a specific mode.

A power switch 100 located in a connector compartment 102 on a rear 103 of the stimulator 10 may control the supply of power from the battery to the electronics of the stimulator 10. The power switch 100 may be maintained in an off position when the stimulator 10 is shipped and stored, i.e., until the stimulator 10 is programmed by a qualified practitioner, prior to being applied to the patient, preventing drainage of the battery. Pod programming connections or connector 104 (e.g., a ten-pin connector) may be located in the connector compartment 102 for mechanically and electrically coupling the stimulator 10 to the pod 30. A detachable compartment cover 106 may be used to cover the compartment 102. On an opposite end of the housing 80, the stimulator 10 may include a pair of protruding electrode pins 130.

Once the stimulator 10 has been programmed and removed from the pod 30, the one or more electrodes 170 (FIG. 2C) have been connected to the stimulator 10 and the patient, and the power switch 100 has been switched to the on position, the “A” button may be pressed by the practitioner for a certain period of time (e.g., 5 seconds or longer) to enable intensity control for a first therapy, then the “B” button may be held or pressed multiple times to control the intensity level of the stimulator 10 for each specific programmed therapy (e.g., 15 specific therapies). The stimulator 10 may provide up to 52 V into 500 Ohms (104 mA) or 84 V into 1000 Ohms (84 mA). The stimulator 10 includes fine control of intensity with the capability of more than 50 incremental intensity steps between 2 V and 52 V. Each press of the “A” button may cause the stimulator 10 to cycle to the next programmed therapy for adjusting the intensity.

Although a pair of buttons 120 are shown, in alternative embodiments, other numbers of inputs, different inputs, and/or a different procedure may be used for enabling intensity control and adjusting intensity control of the stimulator 10 for each therapy. For example, the “A” and “B” buttons may be pressed in a unique combination to enable intensity control, to control intensity level for each therapy, to put the stimulator 10 in a manual mode, and/or to put the stimulator 10 in an automatic mode.

The inventors have determined that requiring a unique input selection procedure to enable and control intensity of the stimulator 10 (i.e., locking the patient out from controlling the intensity) makes it convenient for the practitioner to adjust intensity levels for each therapy and prevents the patient from manually adjusting the intensity of the electrical signals to an ineffective and/or dangerous level. In an alternative embodiment, if the practitioner is comfortable with giving the patient control of the intensity of the stimulator, the practitioner may disclose the unique input selection procedure to the patient to allow the patient to enable and control intensity of the stimulator 10.

Once the intensity level of each therapy is adjusted to a desired level, the stimulator 10 may be put back into an automatic mode through a unique keypress input selection (e.g., the “A” button may be held for 10 seconds or longer), activating each therapy at their respective programmed intervals until the therapy terminates or repeats.

With reference to FIG. 2E, a block diagram of an embodiment of the main electrical components of the stimulator 10 of FIG. 2A is shown. The stimulator 10 is powered from the very small 3V lithium coin cell battery 92. Because the controller firmware executes when powered, the battery switch 100 is used to preserve battery life until the stimulator 10 is programmed and ready for use. The stimulator 10 is interfaced with the pod 30 through the multi-contact pod programming connections 104 and is programmed externally by the programming pod 30. An embedded microcontroller 142 programmed by the external pod 30 contains flash (program or operating) memory, EEPROM therapy memory, and an internal oscillator for pulse recharge and output control, which is determined by each programmed therapy. In alternative embodiments, other types of persistent, modifiable memory other than the flash memory and the EEPROM may be used or other types of memory may be used.

Because the flash memory and EEPROM memory are reprogrammable by the computer 40 via the pod 30, not only can the EEPROM memory of the stimulator 10 be reprogrammed for different therapies and different therapy combinations, but the operating software in the flash memory may also be easily reprogrammed, eliminating the need to purchase a new stimulator 10 for programming software upgrades. Multiple unique firmware executables (standard, special, or custom) may be made available to qualified practitioners, selected by application software on the computer 40, and programmed into flash memory of the stimulator 10 through the pod 30. Each unique firmware executable may include its own protocol for sequence of therapies and/or intensities. Executables may provide additional flexibility of programmed parameters over those described herein (e.g., may allow for pseudo-random waveforms and intensities). If all aspects of programmability can not be maintained in one executable due to limits on EEPROM or flash memory, multiple executables (or custom executables) may be separately programmed into the stimulator 10 as needed (e.g., one executable may support multiple TENS and EMS therapies, another executable may support pseudo-random uni-polar or bi-polar stimulation signaling and possible fixed pseudo-random excitation). Operating software upgrades can also be easily made to the stimulator 10 through the pod 30.

Both positive 144 and negative 146 charge control circuits utilize switching boost converter topologies which can achieve up to 100 V (no-load) of capacitively stored charge energy. Each circuit 144, 146 utilizes very small surface mount inductors, capacitors, resistors and switching bipolar and MOSFET transistors to achieve extremely small circuit size. The amount of charge is determined by the programmed charge intensity for each programmed therapy. Positive pulse control 148 and negative pulse control 152 circuits deliver output pulses to a charge combining circuit 154, as determined by each programmed therapy. The output pulses are delivered to the two male pin electrode pins 132, to which electrodes 170 (e.g., standard or custom skin surface electrodes) may be attached. Once the stimulator 10 is programmed by the programming pod 30, the mode button “A” and the auxiliary Button “B” may be used to select and set the intensity of each programmed therapy.

With reference to FIG. 2C, in an exemplary embodiment, the electrodes 170 are patient-approved, self-adhesive conductive electrodes. Each electrode 170 may include a connector sleeve 132 that slidably receives the electrode pin 130 of the stimulator 10. The sleeve 132 is connected via a lead wire 180 to a distal self-adhesive electrode patch 190. The distal self-adhesive electrode patch 190 may be affixed to the patient's skin. The stimulator 10 sends electrical stimulation waveform signals into the muscles and nerves though the one or more electrodes 170.

In alternative embodiments, other types of electrodes may be used. For example, but not by way of limitation, in another embodiment of the invention, the practitioner programmable stimulator 10 may be physically applied to the patient's body by way of a custom patient-approved adhesive pouch or holster with the electrode(s) at or near the area of pain or discomfort. Wireless snap electrodes or the like may fit onto the electrode pins 130 of the stimulator 10. In this embodiment, the electrode pins 130 may have a different configuration and/or be located at a different location on the housing 80 than that shown in FIG. 2A. The wireless snap electrodes may be self adhesive and may include a pouch, holster, or other carrying mechanism configured to carry the stimulator 10. The wireless snap electrodes, which carry the stimulator 10, are applied to the patient's skin over the area of pain or discomfort with the stimulator 10. The unique, small, lightweight construction of the practitioner programmable stimulator 10 allows the stimulator 10 to be applied to the local area of pain or discomfort on the patient's body with the electrodes.

With reference to FIG. 2D, the pod 30 may include a generally rectangular, block-shaped base 200 with a generally rectangular, block-shaped recess 202 on an upper surface 204 that forms a stimulator bay 210 sized and shaped to receive the stimulator 10. The simulator bay 210 may include a bottom surface 212, a front wall 214, a rear wall 216, a left-side wall 218, and a right-side wall 220. The bottom surface 212 may include pod programming connections 222 (FIG. 2F) near the front wall 214 that mate with the pod programming connections 104 of the stimulator 10 when the stimulator 10 is docked with the pod 30 to form a communication connection. The rear wall 216 may include electrode pin contacts 224 that receive the electrode pins 130 of the stimulator 10. A front panel 226 of the pod 30 may include one or more status indicators 228 (e.g., one or more of the same or different colored LEDs) to indicate the status of the pod 30. A rear panel (not shown) of the pod may include an on/off switch, a power input, and a communications connection to communicatively couple the pod 30 to the computer 40. The base 200 of the pod 30 may rest on fixed supports 230 extending from an undersurface of the base 200.

With reference to FIG. 2F, a block diagram of an embodiment of the main electrical components of the pod of FIG. 2D is shown. The pod 30 is powered by an AC to DC converter 232. Power from the converter 232 to the pod 30 may be controlled with an on/off switch 234 or may be controlled on/off by sensing data communication from the host computer (e.g., USB activity) and regulated by a linear DC-to-DC Regulator 236, providing DC power to the pod electronics. The computer 40 interfaces to the pod 30 over serial (USB or RS232) or parallel (LTP) physical protocols. A microcontroller 238 manages software protocol from the computer 40 and controls programming of the stimulator 10. A digital to analog converter (“DAC”) 240 converts the signal and supplied it to the positive supply circuit 241 for controlling the programming voltage(s) to the stimulator 10. A digital buffer 242 and pod programming driver circuits 244 buffer the programming signals to and from the stimulator 10. An analog-to-digital converter (“ADC”) 246 and analog conditioning circuits 248 provide measurements of the stimulator battery 92 and electrode output voltages from the pod programming connections 250 and the electrode pin contacts 224.

Through the pod programming connections 222 (FIG. 2F), the pod 30 may be used to program the stimulator 10, supply power to the stimulator 10 while the simulator 10 is docked, measure battery power, transmit and receive information in addition to programming information (e.g., diagnostic, verification, stimulator history such as the number of times the stimulator 10 has been turned on and off, checksum discrepancies).

With reference to FIG. 5, the EEPROM of the embedded microcontroller 142 is programmed by the external pod 30 for storing parameters related to each programmed therapy. In an exemplary embodiment, the EEPROM may include an array of 64 registers of 16 bit (8 bits (MSB) +8 bits (LSB)) memory locations, each therapy utilizing 8 bytes or 64 bits. The table of FIG. 5 shows where the parameters for a particular therapy may be stored in memory. At an array location of Index+0, the THERAPY TYPE parameter is stored in the first 8 bits (MSB 0) of the register and the THERAPY TIME parameter is stored in the second 8 bits (LSB 0) of the register. At an array location of Index+1, the IDLE COUNT parameter is stored in the first 8 bits (MSB 1) of the register and the PULSE PERIOD parameter is stored in the second 8 bits (LSB 1) of the register. At an array location of Index+2, the PULSE STYLE parameter is stored in the first 8 bits (MSB 2) of the register and the RECHARGE CONFIG is stored in the second 8 bits (LSB 2) of the register. At an array location of Index+3, the RECHARGE COUNT parameter is stored in the first 8 bits (MSB 3) of the register and the CHECK SUM parameter is stored in the second 8 bits (LSB 3) of the register.

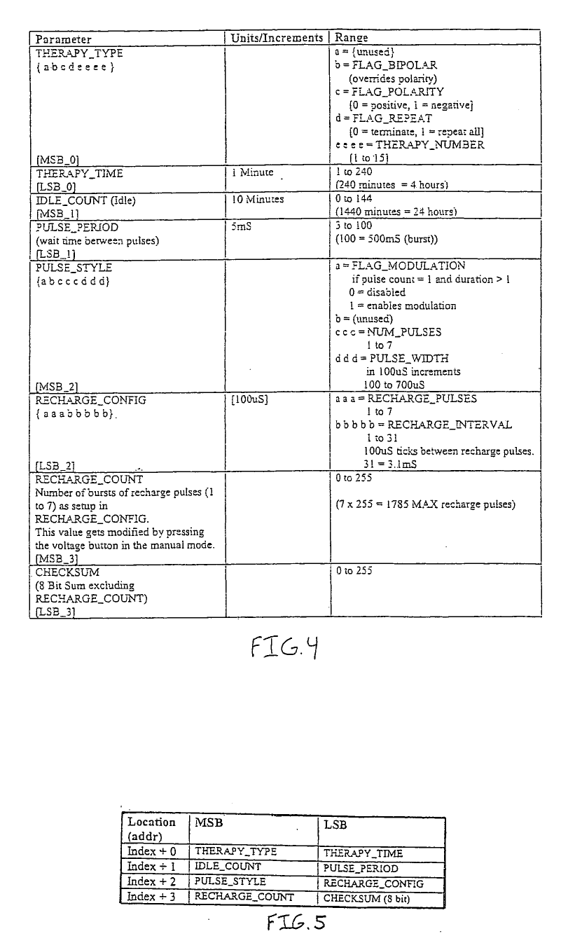

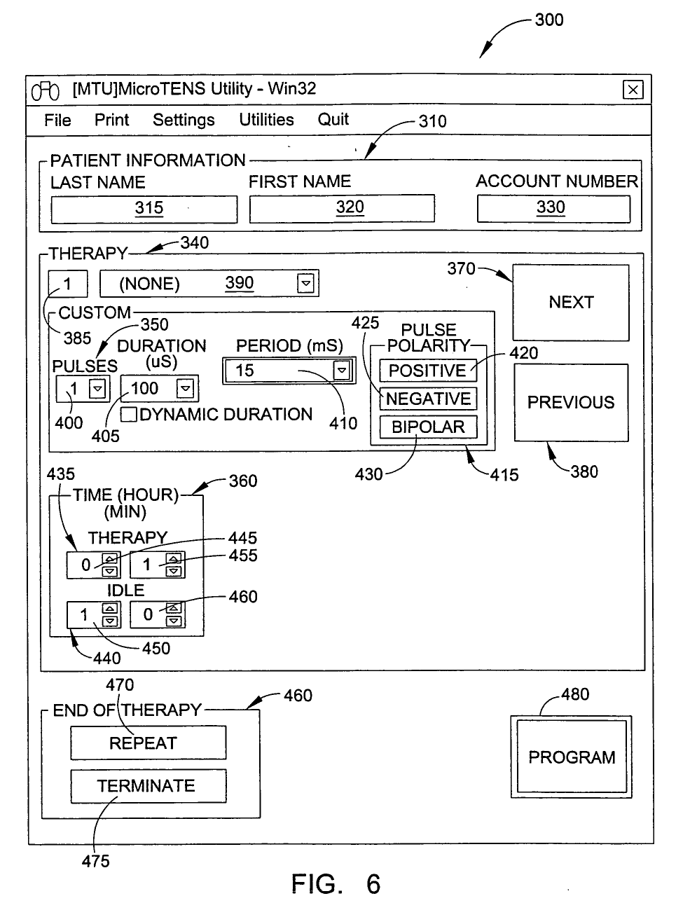

With reference to FIG. 4, the different therapy parameters that may be stored in persistent, modifiable memory of the EEPROM of the stimulator 10 will now be described in more detail.

The THERAPY TYPE parameter indicates the type of electrical stimulation therapy the stimulator 10 is to perform with the b bit indicating whether bipolar treatment should be performed (if bipolar treatment is to be performed, this overrides the c bit described below), the c bit indicating whether the polarity is positive or negative, the d bit indicating whether the therapy should be repeated after it is performed or stop after it is performed), and the four eeee bits indicating which of the 15 specific therapy types is to be performed.

The THERAPY TIME parameter indicates the duration of the therapy in 1 minute increments and ranges from 1 minute to 240 minutes (4 hours).

The IDLE COUNT parameter indicates the duration that the stimulator should remain idle after a therapy is performed in 10 minute increments and ranges from 0 minutes to 1440 minutes (24 hours).

The PULSE PERIOD parameter indicates what the delay or wait count (FIG. 3) should be between pulses or bursts of pulses in 5 mS increments and ranges from 3 to 100 (500 mS).

The PULSE STYLE parameter indicates the characteristics of the pulse(s) that the pulse generator of the stimulator 10 is to perform with the a bit indicating whether modulation is enabled (To be enabled, the pulse count must be 1 and the duration must be greater than 100 uS. For example, if the pulse width is 300 uS and modulation is enabled, the pulse width will change from 300 uS, then 200 uS, then 100 uS, back to 300 uS and repeat through the completion of the therapy time.), the three ccc bits indicating the pulse count or number of pulses (FIG. 3), and the three ddd bits indicating the pulse width or duration (FIG. 3) in 100 uS increments from 100 to 700 uS.

The RECHARGE CONFIG parameter indicates the characteristics of the 17 uS pulse(s) that used to recharge the pulse generator of the stimulator 10 with the three bits aaa indicating the number of recharging pulses (1-7), and the five bits bbbbb indicating the number of 100 uS time duration increments or recharge intervals (FIG. 3) between recharge pulses.

The RECHARGE COUNT parameter indicates the number of bursts of recharge pulse(s) and ranges from 0-255.

The CHECKSUM parameter allows the processor to validate the data contained in the memory block (MSB 0, LSB 0, MSB 1, LSB 1, MSB 2, LSB 2). For example, the processor can read the data from the memory block and perform a predetermined algorithm on the [bits that represent the] data. The result of this algorithm can then be compared to the value in the checksum field. If the value in the checksum field matches the result of the algorithm, then the data that was read from the memory block is valid.

With reference to the software application of FIGS. 6-8, the dialog box 300 used for programming the stimulator 10 via the pod 30 will now be described. The dialog box 300 includes a Patient Information section 310 for identifying the patient and the patient account number that the stimulator therapy data pertains to. The Patient Information section 310 includes a Last Name text field 315 and a First Name text field 320 for inputting the patient's Last Name and First Name respectively. The Patient Information section 310 also includes an Account Number text field 330 for inputting the patient's account number.

The dialog box 300 includes a Therapy section 340 with a Custom sub-section 350, a Time sub-section 360, a Next button 370, and a Previous button 380. At the top of the Therapy section 340 is a therapy number field 385 that identifies which numbered therapy of the sequence of consecutively numbered therapies (e.g., therapies 1-16, therapies 1-64). To the right of the therapy number field 385 is a therapy type field 390 with a drop-down menu of different therapy types. The Custom sub-section 350 includes a Pulses field 400, a Duration (uS) field 405, and a Period field (mS) 410. All of these fields 400, 405, 410 include drop-down menus for selecting a value to be entered in the fields. The Custom sub-section 350 also includes a Pulse Polarity section 415 where a Positive button 420, a Negative button 425, or a Bipolar button 430 may be actuated. The Time sub-section 360 includes a Therapy section 435 and a Idle section 440 where time may be incrementally increased or decreased in an hour field 445, 450 and a minute field 455, 460. Up and down arrow buttons in the Time sub-section 360 allow the practitioner to adjust the hour and time.

The dialog box 300 also includes a End of Therapy section 465 with a Repeat button 470 and a Terminate button 475.

A Program button 480 in the lower-right corner may be actuated to download the input and selected data to the stimulator 10 via the pod 30.

A method of programming the stimulator 10 will now be described. The stimulator 10 is docked with the pod 30 as described above and the pod 30 is communicatively coupled to the computer 40 (preferably a personal computer) through the connection 50. The computer 40 is turned on and a software application is run on the computer 40 to bring up the dialog box 300. The software application is used to program and verify accurate programming of the EEPROM memory in the stimulator 10 while the stimulator 10 is docked in the pod 30. The dialog box 300 is used to program or prescribe an electrical stimulation therapy for the patient into the stimulator 10. The name of the patient is entered in the name fields 315, 320 and the account number of the patient is entered in the account number field 330.

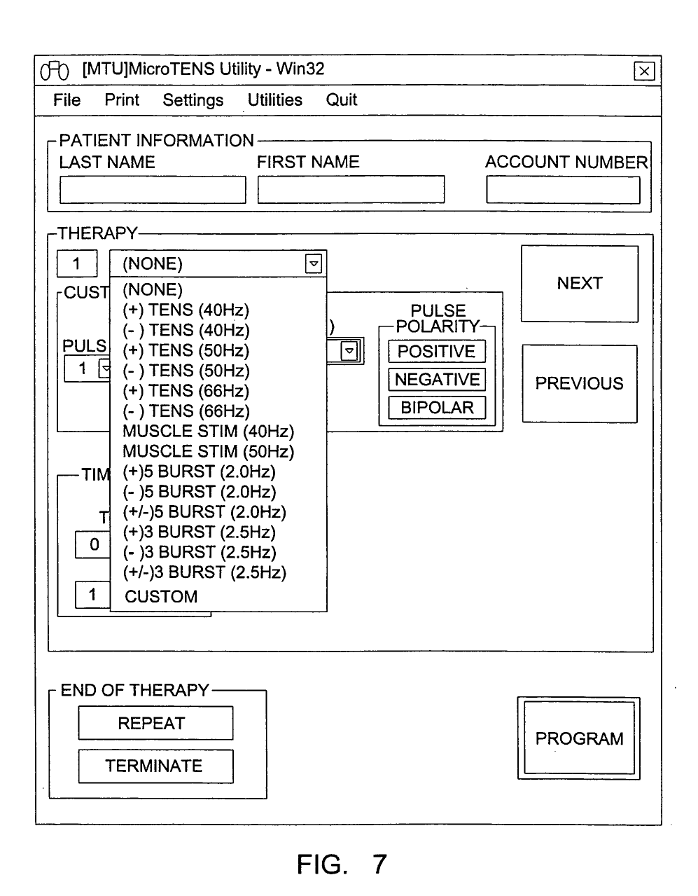

As shown in FIG. 7, the practitioner opens the drop down menu for therapy type field 390 and selects one of the therapy types (e.g., TENS, Muscle Stim (EMS), Burst, Custom) for the first therapy. Each therapy determined in turn by the practitioner may be unique, or they may all be the same with different intensity settings set manually by the practitioner after programming, or a combination of both. In may cases, only a single therapy may be programmed, but the capability for multiple therapies and custom therapies is always available to the practitioner. The practitioner may select a single simple predetermined therapy from the list of therapy types or may select Custom and enter the parameters for a custom therapy.

A therapy type not shown in the drop down menu in FIG. 7 that may be programmed into the stimulator 10 is a micro-current therapy waveform for edema reduction or other complications. This therapy may include a very low current/intensity output in a pseudo-random bi-polar stimulation waveform. It has been determined that the most effective treatment yielding long-term results is subsensory. Such treatments are characterized by extremely low frequencies (0.3 to 0.8 Hz) and intensities, and a bi-phasic (±) waveform. In am embodiment of a micro-current stimulator, the current level may range from 10 to 600 microamps. In preferred embodiment, these treatments use less than 1/100 of the current levels of conventional electrical muscle stimulation (“EMS”). In a more preferred embodiment, these treatments use less than 1/500 of the current levels of conventional EMS. In a most preferred embodiment, these treatments use about 1/1000 of the current levels of conventional EMS. The output stimulation waveform may be “pseudo-random” in that it may be periodic (e.g., 20 seconds on, alternating with 2 seconds off, over a duration of time or continuously), but in that on time the bi-polar output pulses have different pulse widths over different time intervals. The micro-current therapy may be a separate version of firmware, loaded via the pod 30 into the stimulator 10.

It should be noted, faster ON/OFF inter-cycle timing may provided for EMS (electrical muscle stimulation) compared to TENS. For example, during the on cycle in an EMS mode (bipolar), the stimulation current may cycle on for up to 10 seconds and off for up to 2 seconds as programmed parameters. Thus, if an EMS mode is programmed to run on for 20 minutes and off for 1 hour, during the 20 minute on period, the stimulation current may cycle on for 2 to 10 seconds and off for 0 to 2 seconds in 1 second (or fractions of a second) programmed increments.

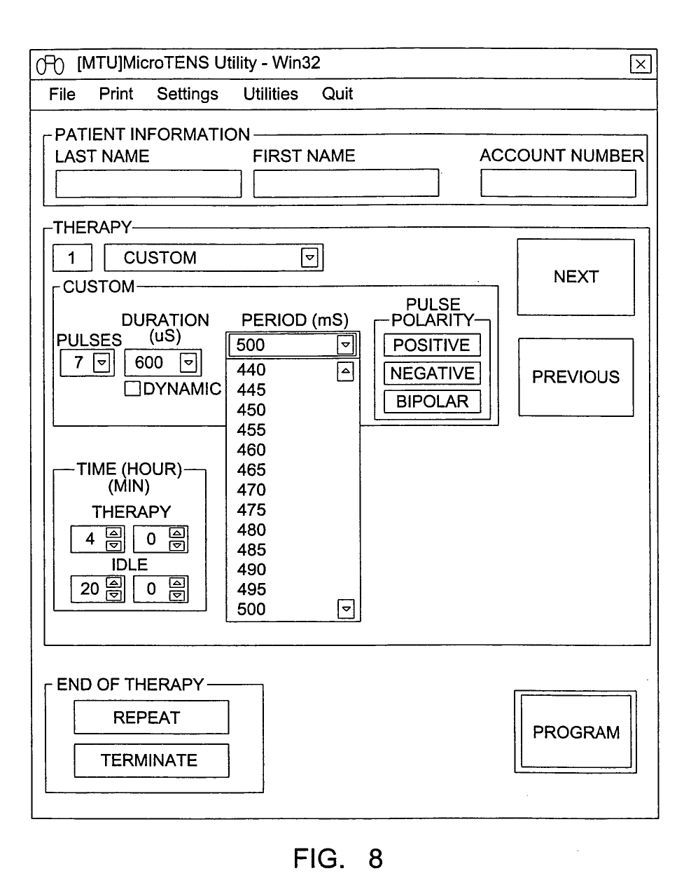

With reference to FIG. 8, if Custom is selected, the fields 400, 405, 410 in the Custom sub-section 350 are activated and the practitioner may select the desired parameters in this section 350. The practitioner selects the number of pulses per wavefront by using the drop-down menu in the pulses field 400, selects the pulse duration by using the drop-down menu in the duration field 405, and selects the time between pulses by using the drop-down menu in the period field 410. The practitioner may then input the pulse polarity by selecting one of the buttons 420, 425, 430 in the pulse polarity section 415.

The overall time duration of the therapy is input using the up and down arrow buttons adjacent the hour field 445 and minute field 455 (or by entering the time in these fields with the keyboard) of the therapy section 435. Similarly, the time between unique therapies or similar therapies is input using the up and down arrow buttons adjacent the hour field 450 and minute field 460 (or by entering the time in these fields with the keyboard) of the idle section 440.

The practitioner may select the terminate button 475 to cause the stimulator 10 to terminate at the end of a programmed session (elapsed minutes, hours or days) or select the repeat button 470 to continuously repeat the programmed session until the battery dies or the patient's next practitioner visit. The practitioner selects the program button 480 to cause the programmed session to be programmed into the persistent, modifiable memory of the stimulator 10. A second therapy session, third therapy session, etc. may be programmed in a like manner by paging between therapy session dialog boxes with the next button 370 and the previous button 380.

FIG. 9 is a block diagram illustrating an exemplary computer 40 as may be used in connection with various embodiments described herein. For example, the computer 40 may be used in conjunction with programming and verifying accurate programming of the persistent, modifiable memory of the stimulator 10 in the manner set forth above. However, other computers and/or architectures may be used, as will be clear to those skilled in the art. Further, the description of many of the elements of the computer 40 described below (e.g., processor 552, main memory 556, secondary memory 558) is applicable to corresponding elements in the stimulator 10 and the pod 30.

The computer 40 preferably includes one or more processors, such as processor 552. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 552.

The processor 552 is preferably connected to a communication bus 554. The communication bus 554 may include a data channel for facilitating information transfer between storage and other peripheral components of the computer 40. The communication bus 554 further may provide a set of signals used for communication with the processor 552, including a data bus, address bus, and control bus (not shown). The communication bus 554 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

Computer 40 preferably includes a main memory 556 and may also include a secondary memory 558. The main memory 556 provides storage of instructions and data for programs executing on the processor 552. The main memory 556 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 558 may optionally include a hard disk drive 560 and/or a removable storage drive 562, for example a floppy disk drive, a magnetic tape drive, a compact disc (“CD”) drive, a digital versatile disc (“DVD”) drive, etc. The removable storage drive 562 reads from and/or writes to a removable storage medium 564 in a well-known manner. Removable storage medium 564 may be, for example, a floppy disk, magnetic tape, CD, DVD, etc.

The removable storage medium 564 is preferably a computer readable medium having stored thereon computer executable code (i.e., software) and/or data. The computer software or data stored on the removable storage medium 564 is read into the computer-40 as electrical communication signals 578.

In alternative embodiments, secondary memory 558 may include other similar means for allowing computer programs or other data or instructions to be loaded into the computer 40. Such means may include, for example, an external storage medium 572 and an interface 570. Examples of external storage medium 572 may include an external hard disk drive or an external optical drive, or and external magneto-optical drive.

Other examples of secondary memory 558 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage units 572 and interfaces 570, which allow software and data to be transferred from the removable storage unit 572 to the computer 40.

Computer 40 may also include a communication interface 574. The communication interface 574 allows software and data to be transferred between computer 40 and external devices (e.g. printers), networks, or information sources. For example, computer software or executable code may be transferred to computer 40 from a network server via communication interface 574. Examples of communication interface 574 include a modem, a network interface card (“NIC”), a communications port, a PCMCIA slot and card, an infrared interface, and an IEEE 1394 fire-wire, just to name a few.

Communication interface 574 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 574 are generally in the form of electrical communication signals 578. These signals 578 are preferably provided to communication interface 574 via a communication channel 576. Communication channel 576 carries signals 578 and can be implemented using a variety of communication means including wire or cable, fiber optics, conventional phone line, cellular phone link, radio frequency (RF) link, or infrared link, just to name a few.

Computer executable code (i.e., computer programs or software) is stored in the main memory 556 and/or the secondary memory 558. Computer programs can also be received via communication interface 574 and stored in the main memory 556 and/or the secondary memory 558. Such computer programs, when executed, enable the computer 40 to perform the various functions of the present invention as previously described.

In this description, the term “computer readable medium” is used to refer to any media used to provide computer executable code (e.g., software and computer programs) to the computer 40. Examples of these media include main memory 556, secondary memory 558 (including hard disk drive 560, removable storage medium 564, and external storage medium 572), and any peripheral device communicatively coupled with communication interface 574 (including a network information server or other network device). These computer readable mediums are means for providing executable code, programming instructions, and software to the computer 40.

In an embodiment that is implemented using software, the software may be stored on a computer readable medium and loaded into computer 40 by way of removable storage drive 562, interface 570, or communication interface 574. In such an embodiment, the software is loaded into the computer 40 in the form of electrical communication signals 578. The software, when executed by the processor 552, preferably causes the processor 552 to perform the inventive features and functions previously described herein.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

It should be noted, although the stimulator 10 is described as a transcutaneous electrical stimulator, the stimulator 10 may be used for faradic, electromagnetic, or other forms of electrical stimulation. Further, the stimulator 10 may be used as a device for electroporation, electrophoresis, iontophoresis, and electrochemical applications.

An important feature of the invention is the limited controllability of the stimulator 10 by the patient. The battery may not be replaceable by the patient and the only input control given to patient related to therapy delivery is the ability to turn the stimulator 10 on or off. Once the battery dies, the stimulator 10 is either disposed of or returned to the office of the practitioner for return to a third party for refurbishing (e.g., environmentally responsible battery replacement, device integrity testing). After refurbishing, the stimulator 10 is available for reprogramming and reuse. Requiring a code (i.e., locking the patient out from controlling the intensity) for enabling intensity control and controlling intensity prevents the patient from manually adjusting the intensity of the electrical signals to an ineffective and/or dangerous level.

Another important feature of the invention is the “prescription” nature of the programmed therapy, in that the practitioner prescribes a specific therapy and the stimulator 10 provides the therapy at the prescribed intervals without patient intervention. If the electrical pulses emitted by the stimulator are uncomfortable to the patient, the patient simply turns the stimulator 10 off with the “A” button (if the “A” button also functions as an on/off switch), removes the stimulator 10, and notifies the practitioner.

A further important feature of the invention is that the stimulator 10 is considerably smaller, lighter, less cumbersome, and less noticeable to the user or patient than stimulators used in the past, increasing the comfort of the patient and increasing the chances that the patient will use the stimulator 10.

It will be readily apparent to those skilled in the art that still further changes and modifications in the actual concepts described herein can readily be made without departing from the spirit and scope of the invention as defined by the following claims.

What is claimed is:

1. A method of programming multiple electrical stimulation therapies into a transcutaneous electrical stimulator at a practitioner location, comprising:
providing at the practitioner location a small, lightweight, portable, external, programmable transcutaneous electrical stimulator that provides multiple electrical stimulation therapies to a patient through the patient's skin, the stimulator including persistent, modifiable memory programmable by a practitioner for prescribing multiple electrical stimulation therapies for the patient;
providing at the practitioner location a programming pod interfaced with the stimulator to program multiple stimulation therapies into the persistent, modifiable memory of the stimulator;
providing at the practitioner location a computer interfaced with the programming pod to program multiple stimulation therapies into the persistent, modifiable memory of the stimulator via the programming pod;
using the computer at the practitioner location to program multiple therapies into the persistent, modifiable memory of the stimulator via the programming pod.

2. The method of claim 1, further including verifying with the stimulator interfaced with the programming pod that the stimulator is operating correctly before applying the stimulator to the patient.

3. The method of claim 1, further including using the stimulator to deliver multiple electrical stimulation therapies to the patient, returning the stimulator to the practitioner, and reprogramming the stimulator through the enumerated steps of claim 1.

4. The method of claim 1, further including using the stimulator to deliver multiple electrical stimulation therapies to the patient, transferring the stimulator to a refurbishing location, and refurbishing the stimulator.

5. The method of claim 1, wherein separately programming each therapy of the multiple therapies includes programming a micro-current therapy waveform into the stimulator suitable for edema reduction, and further including using the stimulator to deliver the programmed micro-current therapy waveform to the patient for edema reduction.

6. The method of claim 1, wherein the stimulator includes one or more practitioner controllable inputs requiring a specific input selection procedure to prevent patient control of all aspects of delivery of the multiple therapies except for on/off control of the stimulator.

7. The method of claim 1, wherein the stimulator includes one or more practitioner controllable inputs that control intensity of the multiple therapies, and the method further includes applying the stimulator to the patient, and the practitioner controlling the intensity of each therapy of the multiple therapies using a specific input selection procedure, which is not known to the patient, involving the one or more practitioner controllable inputs.

8. A computer readable medium having stored thereon one or more sequences of instructions for causing one or more microprocessors to perform the steps for programming multiple electrical stimulation therapies into a transcutaneous electrical stimulator via a programming pod, the steps comprising:
selecting a specific therapy to be programmed out of multiple sequential electrical stimulation therapies;
selecting one of multiple different types of predetermined therapies and a custom therapy;
selecting a type of polarity for the stimulator;
selecting a time duration of the therapy;
selecting an idle time duration between therapies;
selecting the next specific therapy to be programmed out of multiple sequential electrical stimulation therapies and repeating the above steps until all the therapies of the multiple sequential electrical stimulation therapies are input;
selecting one of a repeat function to cause the stimulator to cycle through multiple therapies then repeat and a terminate function to cause the stimulator to cycle through multiple therapies then stop; and
selecting a program function to cause the programming pod to program the multiple electrical stimulation therapies into the transcutaneous electrical stimulator.
